(19) United States
(12) Patent Application Publication
(10) Pub. No.: US 2023/0135367 A1
(43) Pub. Date: May 4, 2023

(54) DETERMINING INFECTION RISK LEVELS

(71) Applicant: International Business Machines Corporation, Armonk, NY (US)

(72) Inventors: Arpita Kushwaha, North York (CA); Lior Aronovich, Thornhill (CA)

(21) Appl. No.: 17/516,740

(22) Filed: Nov. 2, 2021

(51) Int. Cl. G16H 50/30; G16H 50/80; H04W 4/02

(52) U.S. Cl. CPC G16H 50/30; G16H 50/80; H04W 4/025

(57) ABSTRACT

A computer-implemented method for determining aggregate health risk factors comprising one or more processors configured for receiving individual profile data comprising individual attributes corresponding to an individual, the individual attributes comprising demographic data, medical data, and individual location data. Further, the computer-implemented method may be configured for determining an individual risk factor based at least on the individual profile data. Responsive to the individual risk factor satisfying a first condition, the computer-implemented method may be configured for generating a first alert indicating the individual risk factor for the individual.

### BACKGROUND

The present invention relates generally to the field of health risk assessment, and more particularly to determining infection risk levels for individuals and locations.

Considering the ongoing global pandemic, the World Health Organization (WHO) Strategic and Technical Advisory Group for Infectious Hazards (STAG-IH) regularly reviews and updates its COVID-19 risk assessment to make recommendations to the WHO health emergencies program. One of the most effective measures to combat COVID-19 is to practice social distancing. The structures of social contact critically determine the spread of the infection, and social distancing measures appear to be a most effective mitigation technique, also in combination with other measures such as vaccines and wearing of masks.

### SUMMARY

Aspects of the present invention disclose computer-implemented methods, computer program products, and computer systems including one or more processors to receive individual profile data comprising individual attributes corresponding to an individual, the individual attributes comprising demographic data, medical data, and individual location data. Further, the one or more processors determine an individual risk factor based at least on the individual profile data. Responsive to the individual risk factor satisfying a first condition, the one or more processors generate an alert indicating the individual risk factor for the individual.

### BRIEF DESCRIPTION OF THE DRAWINGS

FIG. 1 depicts a block diagram of a distributed data processing environment for determining an aggregated health risk, in accordance with an embodiment of the present invention;

FIG. 2 depicts a block diagram of a system for determining an aggregated health risk, in accordance with an embodiment of the present invention;

FIG. 3 depicts a flowchart of a computer-implemented method for determining an aggregated health risk, in accordance with an embodiment of the present invention; and

FIG. 4 depicts a block diagram of a computing device of distributed data processing environment, in accordance with an embodiment of the present invention.

### DETAILED DESCRIPTION

Infection risk levels associated with an individual or a location are not accurately assessed due to lagging technologies in times of a greater need for accuracy and timeliness.

Embodiments described herein recognize that current solutions implemented to assess health risk factors rely heavily on self-reporting or self-reported data and those solutions are based primarily on outdated data and confidence in the data. To assist in self-reporting methods, home-based testing kits are available for patients to use at their convenience and allow the testing samples to be reported back to a clinical lab for verification. However, home-based testing kits are not readily available to all due to cost to procure and government regulations. Furthermore, the time it takes to return the testing kit and to obtain the results may be too late to adequately address a positive test situation and limit the probability of infecting others.

Embodiments described herein also recognize that, to ease the process of assessing health risk factors, embodiments of the present invention are configured to collect health risk factor data and process the data to determine a level of risk associated with an individual, location, or both.

Embodiments of the present invention describe computer-implemented methods, computer systems, and computer program products to determine and assess health risk factors for locations based on location data corresponding to a positive case (e.g., a positive test for an infectious disease) at a location and to determine and assess health risk factors for individuals based on individual profile data. Assessing health risk factors may include computing a probability of infection based on a computation performed on an individual level and on a location level. The health risk factor may then be used to trigger different actions like alerts and extra sanitation effort for that location or individual to help reduce the risk of other people within proximity of the location or individual from becoming exposed to the infectious disease. Embodiments described herein mobilize comprehensive information to compute infection probability as a valuable tool, not only to prevent and control a pandemic outbreak, but also accessible and affordable for self-diagnosis.

Embodiments of the present invention provide one or more processors configured for determining an infection risk for an individual. For example, individual profile data (i.e., data corresponding to an individual person) may be processed by specially designed algorithms described herein to determine a risk level score for the individual. Individual profile data may include demographics (e.g., age, race or ethnicity, gender, profession), medical information (e.g., pre-existing medical conditions, current symptoms), and locale information (e.g., physical exposure to high-risk individuals, location/travel history). Thus, all individual profile data may be factored into estimating an individual's infection risk.

Embodiments of the present invention provide one or more processors configured for applying an attribute weight to each corresponding attribute represented in the individual profile data. Each attribute may have a corresponding weight based on the attribute's degree of influence on the infection risk level. For example, medical information attributes may have greater weights than demographics attributes, or location information attributes may have greater weights than medical information. Furthermore, specific attributes within a broader category of attributes may have varying weights among the attributes group. For example, within the demographic attributes group, an age attribute may have a greater weight than a profession attribute if the individual is older than a threshold age (e.g., 65 years old) and works in a low-infection risk environment (e.g., Accountant).

Even further, embodiments described herein may include attributes having weights that vary depending on the individual's life circumstances. For example, an individual may be employed in a low-risk profession (e.g., isolated Warehouse Clerk) during a first time-period and then gain new employment in a high-risk profession (e.g., Nursing Assistant) at a later time-period, resulting in the weight applied to the profession attribute to change accordingly. However, some weights may remain constant as the attribute is static and not dynamic. For example, the weight for race or ethnicity attributes are static for the individual because the individual's racial identity is largely considered to be one that does not change over the life of the individual. Once the weights are applied to the attributes, the total infection risk may be determined as an aggregation of the weighted attribute risk levels for each of the individual attributes. In other words, the unified risk factor for the individual may be determined based on an aggregate of the weighted individual attributes represented in the individual profile data.

Embodiments of the present invention provide one or more processors configured for determining an infection risk level for a location. Infection risk for a location may be estimated based on location data at a geographical location. Estimating location-based infection risk may be determined based on location-based factors and be processed by specially designed algorithms described herein. For example, location-based factors may include local individual risk level (e.g., risk level of individuals that are within proximity of (e.g., visiting, residing at) the location), location function (e.g., medical, retail, grocery, municipal, corporate, social, transportation, dining) location time duration (e.g., amount of time at the location), location size (e.g., small, medium, large), location density (e.g., maximum occupancy vs. current occupancy), location surface contact frequency (e.g., high contact, medium contact, low contact), location air access (e.g., enclosed, open), and location sanitization (e.g., sanitizing stations, air filtration).

Embodiments of the present invention provide one or more processors configured for determining a local individual risk level based on an aggregate of individual risk factors at the location. For example, local individual risk level may be based on a combination of individual risk level scores at the location as described above herein. Further, local individual risk level may be based on an aggregate of individual risk level scores at the location, wherein the local individual risk level may be determined when each of the individuals are within proximity of the location during the same time frame.

Embodiments of the present invention may be configured for determining a location function (e.g., medical, retail, grocery, municipal, corporate, social, transportation, dining). In an embodiment, locations may have a function that attracts individuals with a higher infection risk level compared to other locations with a function attracting individuals with a lower infection risk level. For example, location functions corresponding to hospitals, clinics, texting facilities, and pharmacies may contribute greater to the location infection risk level because infected patients attend those locations for treatment. In contrast, location functions corresponding to grocery stores or restaurants may contribute lesser to the location infection risk level because individuals are more likely no infected or are not experiencing infection symptoms while attending those locations.

Embodiments of the present invention may be configured for determining a location time duration (e.g., amount of time at the location). For example, as more individuals interact with the location, as the risks calculated for these individuals are higher, and as individuals spend more time in the location—the time duration factor increase the risk from individuals for the location.

Embodiments of the present invention may be configured for determining a location size (e.g., small, medium, large). For example, smaller locations enable infections to spread more easily because people are physically closer. For example, smaller locations increase the infection risk throughout the location because individuals present within the location are physically closer to each other.

Embodiments of the present invention may be configured for determining a location density (e.g., maximum occupancy vs. current occupancy). For example, a location having a density that exceeds a predetermined threshold may increase the infection risk because more dense locations limit the ability of individuals to socially distance.

Embodiments of the present invention provide one or more processors configured for determining a location surface contact frequency (e.g., high contact, medium contact, low contact). For example, a location having a high surface contact frequency increase the infection risk compared to a location having a low surface contact frequency because unsanitized surface contacts retain infectious particles for an extended period, making the infectious particles commutable to other individuals who touch the infected unsanitized surfaces. In other words, the higher the degree by which individuals touch the same surfaces and/or breathe uncirculated/unfiltered air in the location, the higher the infection risk at the location.

Embodiments of the present invention provide one or more processors configured for determining a location air access (e.g., enclosed, open). For example, enclosed locations have a greater infection risk compared to open locations because enclosed locations have limited air access for individuals to access fresh air free from infectious particles emitted from contagious individuals.

Embodiments of the present invention provide one or more processors configured for determining a location sanitization (e.g., sanitizing stations, air filtration). In an embodiment, one or more IoT devices may be configured to sanitize surface contacts or to provide sanitizer to individuals prior to, or while within the location. In another embodiment, one or more IoT devices may be configured to sanitize the air by providing air filtration to the air flowing throughout the location.

Embodiments of the present invention may be configured for determining the individual risk factor by calculating an age risk factor based at least on a positive case quantity for an age group in a region, an age group population for the age group in the region, wherein the individual risk factor may include a first individual score based on the positive case quantity and the age group population.

Embodiments described herein include computer-implemented methods to identify users within a particular open space environment, wherein each user may provide information or be provided with information to create a user profile. Information used to create a user profile may include demographic data, biometric data, health data, activity data, or other types of data corresponding to the user and/or the associated user device.

Embodiments described herein may be configured to receive all the relevant data at a machine learning model, process the relevant data, and generate model output data corresponding to estimating infection health risks for one or more of an individual and a location. Further, embodiments described herein may be configured provide alerts to individuals navigating the location about the estimated risk factor.

IoT device data gathered from the IoT devices within the location may also be processed to determine the state or status of the IoT devices and which actions the IoT devices are configured to perform. Action may include providing notifications (e.g., audible, luminous, haptic) to alert users navigating the location of the health risk factors.

In an embodiment, a location may be identified where one or more areas of a geographic location. Further, the location may be associated with one or more IoT devices (e.g., microphone, temperature sensor) configured to detect health risk activity (e.g., sounds, temperature changes) within range of the one or more IoT devices. Data corresponding to detected activity may be processed by a risk factor analysis model to determine a risk factor score for each location and trigger follow up actions.

In an embodiment, data (e.g., sensor data) corresponding to the location may be gathered by the IoT devices active in the location. The gathered data may be processed by the risk factor analysis model. The gathered data may include sound data (e.g., coughs, sneezes, vocal projections) corresponding to excessive release of aerosolized particles may be processed for each section of the open space environment. For each sound corresponding to an elevated health risk detected, the one or more processors may be configured to generate an alert to notify other users within, or custodians of, the location about the health risk event that has occurred including the location of which the health risk event occurred. Further, the gathered data may include temperature data (e.g., change in ambient temperature, elevated user body temperature) corresponding to a user that may be suffering from a fever related to an infectious health condition.

In an embodiment, the one or more processors may be configured to determine (e.g., calculate) a risk factor score based on the sensor data. For example, when an event (e.g., health risk event) is detected, the sensor data is transmitted to the risk factor analysis model and processed to update (e.g., increment by a predetermined amount) the risk factor score for the section where the event was detected. For example, if the event that was detected in a first section is a cough event, then the risk factor score for the first section may be incremented by a predefined value (e.g., 5 for a cough event, 7 for a sneeze event).

In an embodiment, the one or more processors may be configured to apply a time weight to the risk factor score, wherein the time weight may be configured to diminish the risk factor score over time. For example, if the risk factor score for a first section of the location is a total of 5 (e.g., 5 for a cough event), and a time weight for the cough event is a reduction of 2 for the first 15 minutes and 3 for the second 15 minutes, then after 30 minutes has expired, the risk factor score for the first section would be 0. Thus, the time weight for the cough event reduces the risk factor score to 0 after 30 minutes as the potency of the aerosolized particles diminishes over time.

The present invention will now be described in detail with reference to the Figures.

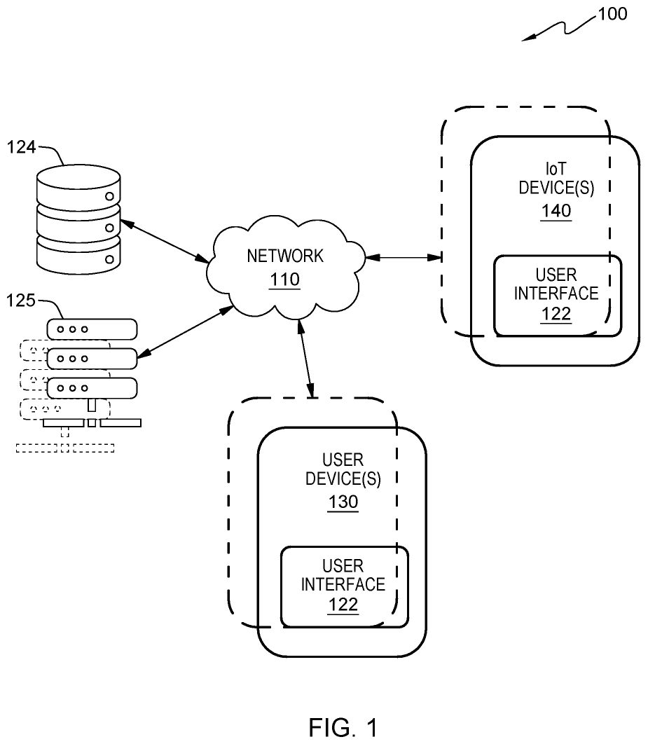

FIG. 1 depicts a block diagram of a distributed data processing environment 100 for determining an aggregated health risk, in accordance with an embodiment of the present invention.

FIG. 1 provides only an illustration of one embodiment of the present invention and does not imply any limitations with regard to the environments in which different embodiments may be implemented. As shown in FIG. 1, the distributed data processing environment 100 for determining an aggregated health risk includes network 110 configured to facilitate communication between database 124, server 125, user device(s) 130 and IoT device(s) 140. In an example embodiment, one or more processors may be configured for receiving individual profile data, positive case location data, location data, and sensor data via network 110. Further, one or more processors may be configured for transmitting output data to user device(s) 130 and IoT device(s) 140 via network 110.

Network 110 operates as a computing network that can be, for example, a local area network (LAN), a wide area network (WAN), or a combination of the two, and can include wired, wireless, or fiber optic connections. In general, network 110 can be any combination of connections and protocols that will support communications between user device(s) 130 and IoT device(s) 140. It is further understood that in some embodiments network 110 is optional and the distributed data processing environment 100 for mitigating open space health risk factors can operate as a stand-alone system, where in other embodiments, network 110 may be configured to enable user device(s) 130 and/or IoT device(s) 140 to share a joint database using network 110.

User interface 122 operates as a local user interface on user device(s) 130 through which one or more users of user device(s) 130 interact with user device(s) 130. User interface 122 may also operate as a local user interface on IoT device(s) 140 through which one or more users of user device(s) 130 interact with IoT device(s) 140. In some embodiments, user interface 122 is a local app interface of a program (e.g., software configured to execute the steps of the invention described herein) on user device(s) 130 or IoT device(s) 140. In some embodiments, user interface 122 is a graphical user interface (GUI), a web user interface (WUI), and/or a voice user interface (VUI) that can display (i.e., visually), present (i.e., audibly), and/or enable a user to enter or receive information (i.e., graphics, text, and/or sound) for or from the program via network 110. In an embodiment, user interface 122 enables a user to transmit and receive data (i.e., to and from the program via network 110, respectively). In an embodiment, user interface 122 enables a user to opt-in to the program, input user related data, and receive alerts.

Database 124 may operate as a repository for data associated with server 125, user device(s) 130, IoT device(s) 140, and other data transmitted within network 110. A database is an organized collection of data. For example, individual profile data may include data associated with a user associated with user device(s) 130. Further, device profile data may include data associated with IoT device(s) 140. Database 124 can be implemented with any type of storage device capable of storing data and configuration files that can be accessed and utilized by either of user device(s) 130 or IoT device(s) 140, such as a database server, a hard disk drive, or a flash memory. In an embodiment, database 124 may be accessed by user device(s) 130 or IoT device(s) 140 to store data associated with user device(s) 130 or IoT device(s) 140. In another embodiment, database 124 may be accessed by user device(s) 130 or IoT device(s) 140 to access data as described herein. In an embodiment, database 124 may reside independent of network 110. In another embodiment, database 124 may reside elsewhere within distributed data processing environment 100 provided database 124 has access to network 110.

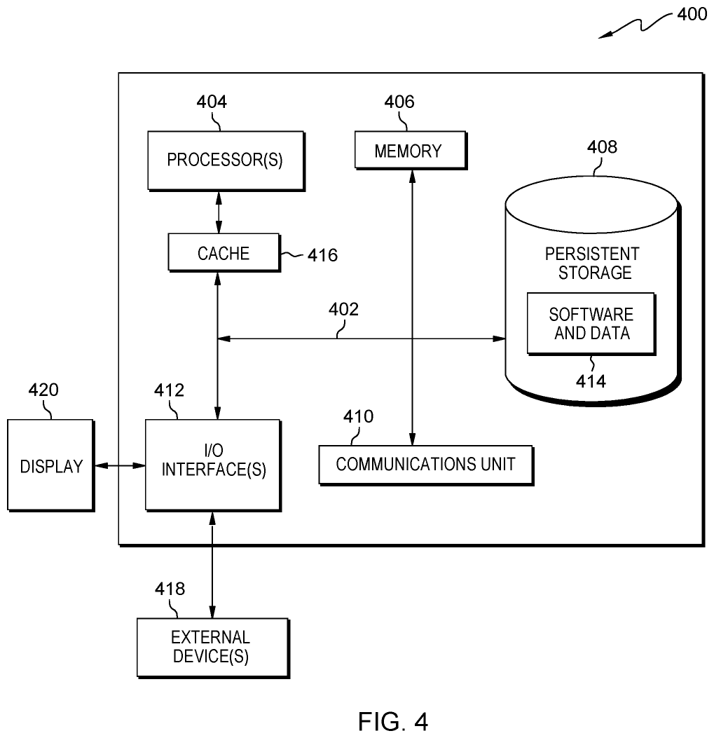

In the depicted embodiment, server(s) 125 may contain a program (e.g., software configured to execute the steps of the invention described herein) and database 124. In some embodiments, server(s) 125 can be a standalone computing device(s), a management server(s), a web server(s), a mobile computing device(s), or any other electronic device(s) or computing system(s) capable of receiving, sending, and processing data. In some embodiments, server 125 may be a laptop computer, tablet computer, netbook computer, personal computer (PC), a desktop computer, a smart phone, or any programmable electronic device capable of communicating with user device(s) 130 and IoT device(s) 140 via network 110. In other embodiments, server(s) 125 represents a server computing system utilizing multiple computers as a server system, such as a cloud computing environment. In yet other embodiments, server(s) 125 represents a computing system utilizing clustered computers and components (e.g., database server computers, application server computers, etc.) that act as a single pool of seamless resources when accessed within distributed data processing environment 100. Server(s) 125 may include components as described in further detail in FIG. 4.

User device(s) 130 may be an electronic device configured for accompaniment with a user. User device(s) 130 may be a personal electronic device such as a mobile communications device, smart phone, tablet, personal digital assistant, smart wearable device, personal laptop computer, desktop computer, or any other electronic device configured for user interaction and gathering individual information to generate an individual profile. User device(s) 130 may include components as described in further detail in FIG. 4.

IoT device(s) 140 may be an electronic device configured to be a component within a smart environment automation system including lighting systems, heating and air conditioning systems, media, and security systems. The electronic device may include a wireless sensor, software, actuators, and computer devices. IoT device(s) 140 may be embedded in mobile devices, industrial equipment, environmental sensors, medical devices, and others. IoT device(s) 140 may be controlled from a remotely controlled system via network 110 or locally controlled system via a local network, or a combination of both. Further, IoT device(s) 140 may be configured to be controlled via a software application installed and executed by IoT device(s) 140. IoT device(s) 140, when connected to a network, may convey usage data and other types of data corresponding to the device itself, or other devices connected via network 110, wherein the data may provide insights that are useful within the scope of the designed application. IoT device(s) 140 may be configured with a processor, memory, and peripherals (not shown) to receive and process data.

For user device(s) 130, a device profile includes, but is not limited to, a user device identifier (ID), a device type (e.g., a smart watch, a smart phone), data usage patterns for user device(s) 130, and data usage models for user device(s) 130. Data usage patterns may include data type, data use frequency, and user device data use history. A device profile may be created for each user device 130 in network 110. User device(s) 130 may consider data usage patterns and data usage models in a device profile when determining whether to execute a data usage request by user device(s) 130.

For IoT device(s) 140, a device profile includes, but is not limited to, an IoT device identifier (ID), and an IoT device type (e.g., a camera, thermometer). A device profile may be created for each IoT device(s) 140 in network 110.

User device(s) 130 and/or IoT device(s) 140 may operate as physical devices and/or everyday objects that are embedded with electronics, Internet connectivity, and other forms of hardware (e.g., sensors). In general, IoT device(s) 140 can communicate and interact with other IoT device(s) 140 over the Internet, or a local network while being remotely monitored and controlled. Types of IoT device(s) 140 include, but are not limited to, smart cameras, smart thermometers, smart locks, garage doors, refrigerators, freezers, ovens, mobile devices, smart watches, air conditioning (A/C) units, washer/dryer units, smart TVs, virtual assistance devices, and any other smart environment devices.

In an embodiment, a user (e.g., individual) may be permitted to opt-in and/or agree to a terms and service agreement upon setting up IoT devices with network 110. The terms and service agreement may document the purpose of the information and data sharing between user device(s) 130 or IoT device(s) 140 and provide access to IoT devices on the network that have been designated for participation in network 110. The user agreement may include all mentioned passing devices that would allow control(s), trigger(s), or action(s) to be executed based on the user's original request. For networks with multiple users and multiple IoT devices, the system may extend the usage agreement to a defined or dynamic group, upon a new user joining said group.

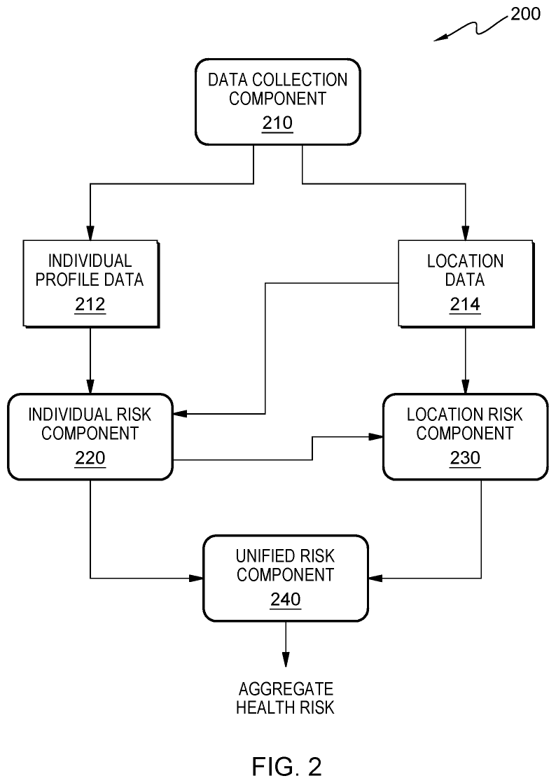

FIG. 2 depicts a block diagram of a system 200 for determining an aggregated health risk, in accordance with an embodiment of the present invention.

In an embodiment, system 200 may include data collection component 210 configured for collecting data corresponding to comprehensive information (e.g., individual profile data 212, location data 214) about positive cases (e.g., positive tests for an infectious disease) of a pathogenic pandemic. For example, location data 214 may include positive case location data representing positive case location attributes about the location. Individual profile data 212 and location data 214 may be collected, stored in, and transmitted from, one or more databases via network 110. Further, data collection component 210 may be configured to identify individual profile data 212 based on individual attributes present in the comprehensive information. Data collection component 210 may be configured to identify location data 214 based on positive case location attributes represented in the comprehensive information. Furthermore, data collection component 210 may be configured to transmit individual profile data 212 to individual risk component 220 and transmit location data to location risk component 230 for further processing.

In an embodiment, system 200 may include individual risk component 220 configured to determine an individual risk factor (It) based at least on individual profile data 212. As mentioned above herein, for example, individual profile data 212 may include demographics, medical information, and location information. Individual risk component 220 may be configured to estimate a risk level score (e.g., individual risk factor (10) for the individual by factoring in numerical representations of the health risks corresponding to the individual attributes.

In an embodiment, individual attributes corresponding to demographics are considered in determining an individual health risk. For example, statistical data representing demographic individual attributes corresponding to age, race, ethnicity, gender, and profession play a crucial role in assessing individuals' infection risk regarding a pathogenic virus may and be used in estimating an individual's health risk.

In an embodiment, individual risk component 220 may be configured to determine an age group infection risk (Rₐ) based on age group demographics represented in the individual profile data. In an embodiment, the age group infection risk (Rₐ) in a location may be based on a number of positive cases (x) for the age group in the location and the age group population (N) in the location. For example, the age group infection risk (Rₐ) may be estimated as a ratio (e.g., x/N) of the number of positive cases (x) and the age group population (N).

In an embodiment, individual risk component 220 may be configured to determine a race or ethnic group infection risk (Re) based on race or ethnic group demographics represented in the individual profile data. In an embodiment, race or ethnic group infection risk (Re) may be based on a number of positive cases (x) for the race or ethnic group in the location and the race or ethnic group population (N) in the location. For example, the race or ethnic group infection risk (Rₑ) may be estimated as a ratio (e.g., x/N) of the number of positive cases (x) and the race or ethnic group population (N).

In an embodiment, individual risk component 220 may be configured to determine a gender group infection risk (R_{g}) based on gender group demographics represented in the individual profile data. In an embodiment, the gender group infection risk (R_{g}) in a location may be based on a number of positive cases (x) for the gender group in the location and the gender group population (N) in the location. For example, the gender group infection risk (R_{g}) may be estimated as a ratio (e.g., x/N) of the number of positive cases (x) and the gender group population (N).

In an embodiment, individual risk component 220 may be configured to determine a profession group infection risk (Rₚ) based on profession demographics represented in the individual profile data. In an embodiment, the profession group infection risk (Rₚ) in a location may be based on a number of positive cases (x) for the profession group in the location and the profession group population (N) in the location. For example, the profession group infection risk (Rₚ) may be estimated as a ratio (e.g., x/N) of the number of positive cases (x) in the profession group and the profession group population (N). For instance, a medical profession group having a duty to serve and treat patients infected with an infectious virus incur a greater risk of infection due to their professional obligations as opposed to a non-medical profession group.

In an embodiment, individual risk component 220 may be configured to determine a medical conditions infection risk (R_{d}) based on medical history represented in the individual profile data. In an embodiment, the medical conditions infection risk (R_{d}) in a location may be based on a plurality of medical conditions (dᵢ), a plurality of severity factors (sᵢ) for the individual, and a plurality of relevance factors (rᵢ) for a particular pandemic. Further, individual risk component 220 may be configured to incorporate a correlation between scientific data and the medical conditions into determining the medical conditions infection risk (Rd). For example, the relevance factor (rᵢ) for a medical condition (dᵢ) may be calculated based on the ratio of people having the condition from the total number of people that tested positive for the pandemic. Therefore, the medical conditions infection risk (R_{d}) for the medical conditions (dᵢ) may be estimated as a ratio (e.g., Σsᵢrᵢ/Σrᵢ) based on the severity factors (sᵢ) and the relevance factors (rᵢ) of the correlated medical conditions (dᵢ).

In an embodiment, individual risk component 220 may be configured to determine a current symptoms infection risk (Rₛ) based on current medical symptoms information represented in the individual profile data. In an embodiment, the current symptoms infection risk (Rₛ) may be based on a plurality of current symptoms (tᵢ), a plurality of severity factors (sᵢ) for the individual, and a plurality of relevance factors (rᵢ) for a particular pandemic. For example, current symptoms (tᵢ) may include throat pain (tₜₚ), cough (t_{cg}), breathing difficulty (t_{bd}), fever (t_{f}), antigens or antibody levels (tₐ), and other relevant symptoms (tₙ). In an embodiment, the relevance factors (rᵢ) for symptoms (tᵢ) may be determined based on statistical data that specifies the significance of a symptom relative to the pandemic, for example the ratio of people having this symptom from the total number of people that tested positive for the pandemic.

In an embodiment, a correlation factor may be determined based at least on statistical data and individual's symptom data. For instance, a matched symptom list may be generated for an individual and the more an individual's symptoms match the matched symptom list, the greater the correlation factor. Therefore, the current medical symptoms infection risk (Rₛ) for the medical symptoms (tᵢ) may be estimated as a ratio (e.g., Σsᵢrᵢ/Σrᵢ) based on the severity factors (sᵢ) and the relevance factors (rᵢ) of the correlated medical symptoms (tᵢ).

In an embodiment, individual risk component 220 may be configured to determine a locale infection risk (Rₗ) based on locale information represented in the individual profile data. In an embodiment, the locale infection risk (Rₗ) may be based on estimated risks of being infected (rᵢ) of people (pᵢ) who are in routine close contact with the individual. For example, such people (pᵢ) may include people in the same household (e.g., family members, household members), at work (e.g., coworkers, colleagues, clients, patients), at school (e.g., classmates, teachers, professors, administrative staff, clerical staff), or other locations the individual routinely visits. Locale infection risk (Rₗ) for an individual may be determined at least based on an estimate of the aggregate of the estimated risks of being infected (rᵢ) of the people (pᵢ) who are in routine close contact with the individual and the number of these people (pₙ) (e.g., Σrᵢ/pₙ).

In an embodiment, physical exposure infection risk (Rex) to high-risk individuals may be based on an individual being within proximity of high-risk individuals or individuals who tested positive for infection. In an embodiment, a high-risk individual is an individual having an individual risk factor exceeding a predetermined threshold (rₜ). For example, if an individual has been within proximity with n number of persons, and m number of persons have individual risk factors (rₜ) exceeding the predetermined threshold (rₜ), then the physical exposure infection risk (Rex) of the individual may be estimated as a ratio (e.g., Σrᵢ/m) of an aggregate risk of high-risk individuals and the m number of high-risk individuals.

In an embodiment, individual risk component 220 may be configured to determine a travel history infection risk (Rₜᵣ) based on travel history information represented in the individual profile data. In an embodiment, the travel history infection risk (Rₜᵣ) may be based on a plurality of regions (gᵢ), a plurality of infection rates associated with the regions (fᵢ), and a plurality of time spent metrics (tᵢ) for an individual respective to the regions. For example, traveling to regions that have high infection rates increases the risk of infection for a person visiting such places. For example, the infection rate for a specific region may be calculated based on the ratio of people who live or have visited the region that tested positive for the pandemic from the total number of people who live or have visited the region. As another example, the values of the time spent metrics (tᵢ) for an individual respective to the regions can be calculated based on the total number of days since the start of the pandemic until the current day (PD) and the number of days that the individual spent in region gᵢ during the timeframe of the pandemic (RDᵢ), as a ratio for example tᵢ=RDᵢ/PD. Therefore, the travel history infection risk (Rₜᵣ) for the regions (gᵢ) may be estimated as a ratio (e.g., Σfᵢtᵢ/Σtᵢ) based on the infection rates associated with the regions (fᵢ) and the time spent metrics (tᵢ) for an individual respective to the regions.

In an embodiment, individual risk component 220 may be configured to consider all factors represented in individual profile data 212 and location data 214 to estimate a total individual infection risk. In an embodiment, the factors are not weighed equally. For example, risk based on travel history may be weighed greater than risk based on gender. In an embodiment, the weights can be calculated from statistical data to reflect how relevant each factor is for determining an overall risk of infection. In an embodiment, mathematical models that enable to calculate weights for features can be used. Therefore, the total individual infection risk may be represented by the following equation: R_{individual}=ΣRᵢwᵢ/wᵢ.

In an embodiment, system 200 may include location risk component 230 configured to determine a location risk factor (R_{loc}) based at least on location data 214 and aggregate individual risk factor (Rₐᵢ) for the individuals at a location. Location data 214 (also referred to as positive case location data) may include positive case location attributes about the location. Positive case location attributes may include local individual risk level (also referred to as aggregated individual risk factor (Rₐᵢ)), location function, location size, location surface contact frequency, and location air access. In an embodiment, a risk level may be determined for each of the positive case location attributes and factored into estimating the location risk factor (R_{loc}). For example, a location may be a neighborhood, wherein the location risk factor (R_{loc}) for the neighborhood is based at least on the aggregated individual risk factor (Rₐᵢ) of all the individuals physically present in the neighborhood and the aggregate risk level for one or more of the location function, location size, location surface contact frequency, and location air access.

In an embodiment, location risk component 230 may be configured to determine an aggregated individual risk factor (Rₐᵢ) for a location based on a plurality of individual risk factors (Rᵢ) for individuals that interact with the location and on a plurality of times (tᵢ) that these individuals spent at the location. For example, as more individuals interact with a location, and as the risks calculated for these individuals are higher, and as individuals spend more time in the location, these factors increase the risk from individuals for the location. The time (tᵢ) spent by an individual in the location can be calculated by dividing the time spent by the individual in the location by the total time period for which the estimation is done. This total time period should be the same for all the individuals for which the calculation is done. The numeric range for each tᵢ is 0 to 1, where 0 indicates that the individual has not spend any time in the location, 1 indicates that the individual has spent all their time in the timeframe in the location, and any number between 0 and 1 correlates to the relative time spent by the individual in the location. Thus, the aggregated individual risk factor (Rₐᵢ) for a location may be estimated as a ratio (e.g., Σᵢ(Rᵢtᵢ)/Σᵢtᵢ) of the total individual risk factor (Rᵢ) for each individual as a product of the time spent at the location and the total time for which the estimation is done.

In an embodiment, location risk component 230 may be configured to determine a location risk (R_{loc}) based at least on a location function (R_{lf}) type risk level. Location function types may include medical, retail, grocery, municipal, corporate, social, park, transportation, dining, or any other type of function, and each function type may be assigned a risk level representative of the function type relative to the other function types. For example, a medical location function type may be assigned a higher risk level due to the greater quantity and frequency of infected individuals drawn to medical locations to seek treatment engaging in physical contact with other individuals at the medical location. An outdoor park function type may be assigned a lower risk level due to the open-air access and limited frequency of individual contact among individuals at the outdoor park. Further, location function type risk level may also be implemented as a transformation on a risk level that is calculated for the location based on other aspects such as for interactions.

In an embodiment, location risk component 230 may be configured to determine a location risk (R_{loc}) based on at least on a location time (Rₗₜ) duration risk level. The location time duration spent by an individual in the location can be calculated by dividing the time spent by the individual in the location by the total time period for which the calculation is done. This total time period should be the same for all the individuals for which the calculation is done. For example, the location function risk based on the location time duration may be determined as an estimate of the ratio (e.g., Σᵢ(Rᵢtᵢ)/Σᵢtᵢ) of the total individual risk factor (Rᵢ) for each individual as a product of the time spent at the location and the total time for which the estimation is done.

In an embodiment, location risk component 230 may be configured to determine a location risk (Rₗₒₜ) based at least on a location size (Rₗₛ) risk level. For example, location size risk level may be assigned to defined risk levels for size classes or ranges of a location, wherein the location size risk function may be based on location size classes, for example R_{loc}=Rₗₛ (size_class). For example, smaller locations can enable infections to spread more easily because people are physically closer. Further, location size risk level may also be implemented as a transformation on a risk level that is calculated for the location based on other aspects such as for interactions.

In an embodiment, location risk component 230 may be configured to determine a location risk (R_{loc}) based at least on a location density (R_{ld}) risk level. For example, location density risk level may be assigned defined risk levels for each density range of individuals for a given fixed area size. For example, higher density can reduce the ability to apply physical distancing and therefore can increase the risk of infection. A density range of individuals for a given fixed area size can be calculated, for example, by calculating an average density of individuals for a given fixed area size and then selecting the density range in which the average density falls in. For example, ranges may include: 1-2 people, 3-5 people, etc. Each of the ranges may be assigned with a representative risk level. Further, an average density of individuals may be calculated by sampling how many individuals are in the location over time, converting each sample to a given fixed size (e.g., per 100 square feet), and then determining an average or another type of metric based on the samples. Furthermore, the number of individuals in multiple separate areas within the location may be samples over time, wherein each sample may be converted to a given fixed size, and an average or another type of metric may be determined based on the samples. Location density risk levels may also be implemented as a transformation on a risk level that is calculated for the location based on other aspects. For example, a transformation on the risk level calculated for interactions.

In an embodiment, location risk component 230 may be configured to determine a location risk (R_{loc}) based at least on a location surface contact frequency (R_{lc}) risk level. For example, the higher the degree by which people touch the same surfaces in the location, the higher the risk of the location. For example, referring to contact surfaces, the average number of individuals touching each of these surfaces may be determined for a fixed timeframe. For example, for a grocery store location, location risk component 230 may be configured to determine the average number of individuals touching each checkout station in an hour. As another example, location risk component 230 may be configured to determine the average number of individuals touching each automated check-in station in a hospital or in an airport. Further, location risk component 230 may be configured to aggregate the average numbers across all surfaces into an aggregated or average number per the timeframe. Thus, location risk component 230 may be configured to assign a defined risk level for each range of the location surface contact frequency metrics. In an embodiment, ranges can be for example: 1-2 individuals per hour in contact with common surfaces, 3-5 individuals per hour in contact with common surfaces.

In yet another embodiment, the overall average number of individuals in the location in a timeframe may be factored in with the location surface contact frequency metrics. For example, location risk component 230 may be configured to determine the ratio of individuals making contact with common surfaces per hour from the total number of individuals in the location per hour. The risk level can be calculated based on this ratio. A similar type of determination may be used for specific areas in the location where air circulation is restricted and/or air is not filtered. Namely, the calculation can be based on the average number of individuals per hour that come into contact with the areas in the location where air circulation is restricted and/or air is not filtered.

In an embodiment, location risk component 230 may be configured to determine a location risk (R_{loc}) based at least on a location air access (Rₗₐ) risk level. For example, location risk component 230 may be configured to assign a risk level for location air access as defined risk levels for enclosed and open location types. For example, enclosed locations tend to present higher risk of infection compared with open locations. Location air access risk levels may be implemented as a transformation on a risk level that is calculated for the location based on other aspects.

In an embodiment, location risk component 230 may be configured to determine a location risk (R_{loc}) based at least on a location sanitization (R_{lz}) risk level. For example, location risk component 230 may be configured to assign a risk level for a location based on local sanitization available at the location. Location sanitization may include sanitizer dispenser stations, air filtration systems configured to sanitize the air circulating at the location. For instance, a location with ample sanitization stations and air filtration systems may be assigned the lowest risk level, whereas a location with no sanitization stations or air filtration systems may be assigned the highest risk level.

In an embodiment, the numeric range of the values of location risk (R_{loc}) may be 0 to 1, where 0 indicates no risk generated from the interactions of individuals with the location, and 1 indicates maximum risk generated from interactions. The calculated risk level for each of these aspects may change over time for a specific location, as the individuals interacting with the location and their risks change over time.

In an embodiment, location risk component 230 may be configured to apply weights (wᵢ) to the infection risk factors, as described above herein regarding the individual attributes.

In an embodiment, location risk component 230 may be configured to determine an aggregated location risk (R_{aloc}) for a location based on the aggregate of the location risk factor (Rₗ) for each attribute associated with the location, as described above herein. In an embodiment, weights for the various attributes can be calculated from statistical data to reflect how relevant each attribute is for determining an overall risk of infection. In an embodiment, mathematical models that enable to calculate weights for features can be used. For example, the aggregated weighted location infection risk may be represented by the following equation: R_{aloc}=ΣRᵢwᵢ/Σwᵢ.

In an embodiment, system 200 may be configured to detect triggers for activating risk calculations or performing risk estimations. For example, triggers for risk calculation for an individual may include a change in the individual attributes, a change in the positive case location attributes, a change in the statistical data collected and used by individual risk component 220 and location risk component 230. Even further, the aggregate health risk may be estimated or calculated for every time period, regardless of whether or not a change occurs in the individual attributes or the positive case location attributes.

In an embodiment, the one or more processors may be configured to gather sensor data from the one or more IoT devices positioned in one or more locations. For example, IoT device(s) may be a microphone configured to capture sensor data corresponding to a cough event detected within a first location. Further, IoT device(s) may be a microphone configured to capture sensor data corresponding to a sneeze event detected within a location, and transmit the sensor data to data collection component 210 for use in determining individual risk health levels and location health risk levels.

In an embodiment, the one or more processors may be configured to determine a risk factor score based on the sensor data for a location. For example, if IoT device(s) in a location detects no events corresponding to an elevated health risk (e.g., coughing, sneezing, voice projections), then the risk factor score will be 0, indicating no health risk is present at that time in that location.

In an embodiment, the one or more processors may be configured to determine one or more events occurred in a location based on the sensor data. The one or more events may include one or more of a cough event, a sneeze event, voice projection event, an infection risk level event, or a temperature event. For example, if the one or more processors process the sensor data and determines that the sensor data corresponds to a coughing sound detected in a location, then the one or more processors may be configured to determine that a cough event has occurred in the location.

In an embodiment, the one or more processors may be configured to update the risk factor score for the location based on the one or more events. For example, if the risk factor score for the location is 0 and a cough event with a risk factor score of 5 is detected in the location, then the risk factor score for the location is incremented by 5 to generate an updated risk factor score of 5. If additional events are detected in the location, then the risk factor scores associated with the additional events are added to the risk factor score for the location accordingly.

In an embodiment, the one or more processors may be configured to apply a time weight to the risk factor score, wherein the time weight may be configured to diminish the risk factor score over time. For example, if the risk factor score for the location is a total of 5 (e.g., 5 for a cough event), and a time weight for the cough event is a reduction of 2 for the first 15 minutes and 3 for the second 15 minutes, then after 30 minutes has expired, the risk factor score for the location would be 0. Thus, the time weight for the cough event reduces the risk factor score to 0 after 30 minutes as the potency of the aerosolized particles diminishes over time.

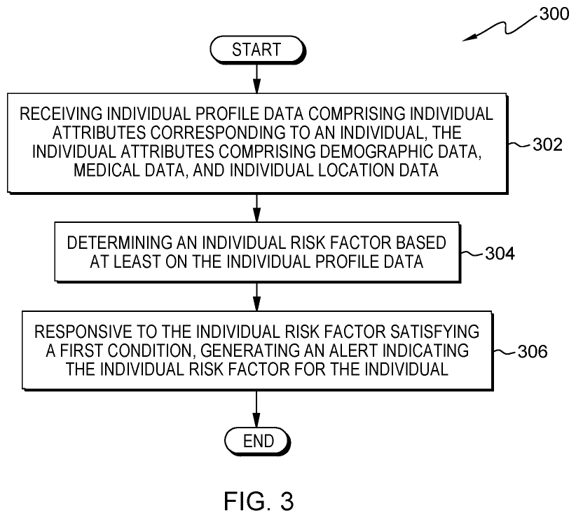

FIG. 3 depicts a flowchart of a computer-implemented method for a flowchart of a computer-implemented method for determining an aggregated health risk, in accordance with an embodiment of the present invention.

In an embodiment, computer-implemented method 300 may include one or more processors configured for receiving 302 individual profile data comprising individual attributes corresponding to an individual, the individual attributes comprising demographic data, medical data, and individual location data.

In an embodiment, computer-implemented method 300 may include one or more processors configured for determining 304 an individual risk factor based at least on the individual profile data. For example, determining 304 an individual risk factor may include one or more processors configured for assigning an individual attribute risk level score to each of the individual attributes, generating a weighted attribute risk level score by applying an attribute weight to each corresponding individual attribute, and aggregating the weighted location risk level score for each of the positive case location attributes to determine the location risk factor.

In an embodiment, responsive to the individual risk factor satisfying a first condition, computer-implemented method 300 may include one or more processors configured for generating 306 an alert indicating the individual risk factor for the individual.

In an embodiment, computer-implemented method 300 may include one or more processors configured for receiving location data corresponding to a positive case at a location, the location data comprising positive case location attributes comprising a local individual risk level, a location function, location size, location surface contact frequency, and location air access; determining a location risk factor for a location based at least on the location data; and responsive to the location risk factor satisfying a second condition, generating a second alert indicating the location risk factor for the location.

In an embodiment, determining a location risk factor may include one or more processors configured for assigning a location risk level score to each of the positive case location attributes, generating a weighted location risk level score by applying a location factor weight to each corresponding positive case attribute, and aggregating the weighted location risk level score for each of the positive case location attributes to determine the location risk factor.

In an embodiment, computer-implemented method 300 may include one or more processors configured for determining a first unified risk factor for the individual at the location based on the individual risk factor and the location risk factor. For example, the first unified risk factor (also referred to as aggregated risk factor) may correspond to some combination of an aggregate individual risk factor and an aggregate location risk factor.

In an embodiment, computer-implemented method 300 may include one or more processors configured for applying a time weight to the local individual risk level, wherein the time weight may be directly proportional to a time duration the individual is present at the location.

In an embodiment, computer-implemented method 300 may include one or more processors configured for detecting a change in one or more of the positive case location attributes and the individual attributes exceeds a predetermined threshold. Further, responsive to detecting the change, computer-implemented method 300 may be configured for determining a second unified risk factor for one or more of the individual risk factor and the location risk factor. Furthermore, responsive to the second unified risk satisfying the condition, computer-implemented method 300 may be configured for generating the alert for one or more of the location and the individual.

FIG. 4 depicts a block diagram 400 of computing device (e.g., user device(s) 130, IoT device(s) 140) of distributed data processing environment 100, in accordance with an embodiment of the present invention, in accordance with an illustrative embodiment of the present invention. It should be appreciated that FIG. 4 provides only an illustration of one implementation and does not imply any limitations with regard to the environments in which different embodiments may be implemented. Many modifications to the depicted environment may be made.

Computing device (e.g., user device(s) 130, IoT device(s) 140) includes communications fabric 402, which provides communications between cache 416, memory 406, persistent storage 408, communications unit 410, and input/output (I/O) interface(s) 412. Communications fabric 402 can be implemented with any architecture designed for passing data and/or control information between processors (such as microprocessors, communications and network processors, etc.), system memory, peripheral devices, and any other hardware components within a system. For example, communications fabric 402 can be implemented with one or more buses or a crossbar switch.

Memory 406 and persistent storage 408 are computer readable storage media. In this embodiment, memory 406 includes random access memory (RAM). In general, memory 406 can include any suitable volatile or non-volatile computer readable storage media. Cache 416 is a fast memory that enhances the performance of computer processor(s) 404 by holding recently accessed data, and data near accessed data, from memory 406.

Software and data 414 may be stored in persistent storage 408 and in memory 406 for execution and/or access by one or more of the respective computer processors 404 via cache 416. In an embodiment, persistent storage 408 includes a magnetic hard disk drive. Alternatively, or in addition to a magnetic hard disk drive, persistent storage 408 can include a solid-state hard drive, a semiconductor storage device, read-only memory (ROM), erasable programmable read-only memory (EPROM), flash memory, or any other computer readable storage media that is capable of storing program instructions or digital information.

The media used by persistent storage 408 may also be removable. For example, a removable hard drive may be used for persistent storage 408. Other examples include optical and magnetic disks, thumb drives, and smart cards that are inserted into a drive for transfer onto another computer readable storage medium that is also part of persistent storage 408.

Communications unit 410, in these examples, provides for communications with other data processing systems or devices. In these examples, communications unit 410 includes one or more network interface cards. Communications unit 410 may provide communications through the use of either or both physical and wireless communications links. Software and data 414 may be downloaded to persistent storage 408 through communications unit 410.

I/O interface(s) 412 allows for input and output of data with other devices that may be connected to server 125, user device(s) 130, and/or IoT device(s) 140. For example, I/O interface 412 may provide a connection to external devices 418 such as a keyboard, keypad, a touch screen, and/or some other suitable input device. External devices 418 can also include portable computer readable storage media such as, for example, thumb drives, portable optical or magnetic disks, and memory cards. Software and data 414 used to practice embodiments of the present invention can be stored on such portable computer readable storage media and can be loaded onto persistent storage 408 via I/O interface(s) 412. I/O interface(s) 412 also connect to a display 420.

Display 420 provides a mechanism to display data to a user and may be, for example, a computer monitor.

The present invention may contain various accessible data sources, such as database 124, that may include personal data, content, or information the user wishes not to be processed. Personal data includes personally identifying information or sensitive personal information as well as user information, such as tracking or geolocation information. Processing refers to any, automated or unautomated, operation or set of operations such as collection, recording, organization, structuring, storage, adaptation, alteration, retrieval, consultation, use, disclosure by transmission, dissemination, or otherwise making available, combination, restriction, erasure, or destruction performed on personal data. Software and data 414 may enable the authorized and secure processing of personal data. Software and data 414 may be configured to provide informed consent, with notice of the collection of personal data, allowing the user to opt in or opt out of processing personal data. Consent can take several forms. Opt-in consent can impose on the user to take an affirmative action before personal data is processed. Alternatively, opt-out consent can impose on the user to take an affirmative action to prevent the processing of personal data before personal data is processed. Software and data 414 may provide information regarding personal data and the nature (e.g., type, scope, purpose, duration, etc.) of the processing. Software and data 414 provide the user with copies of stored personal data. Software and data 414 allow the correction or completion of incorrect or incomplete personal data. Software and data 414 allow the immediate deletion of personal data.

The present invention may be a system, a computer-implemented method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or obj ect code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of computer-implemented methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A computer-implemented method, comprising:
receiving, by one or more processors, individual profile data comprising individual attributes corresponding to an individual, the individual attributes comprising demographic data, medical data, and individual location data;
determining, by one or more processors, an individual risk factor based at least on the individual profile data; and
responsive to the individual risk factor satisfying a first condition, generating, by one or more processors, a first alert indicating the individual risk factor for the individual.

2. The computer-implemented method of claim 1, further comprising:
receiving, by one or more processors, location data corresponding to a positive case at a location, the location data comprising positive case location attributes comprising a local individual risk level, a location function, location size, location surface contact frequency, and location air access;
determining, by one or more processors, a location risk factor for the location based at least on the location data; and
responsive to the location risk factor satisfying a second condition, generating, by one or more processors, a second alert indicating the location risk factor for the location.

3. The computer-implemented method of claim 2, wherein determining the location risk factor further comprises:
assigning, by one or more processors, a location risk level score to each of the positive case location attributes;
generating, by one or more processors, a weighted location risk level score by applying a location factor weight to each corresponding positive case location attribute; and
aggregating, by one or more processors, the weighted location risk level score for each of the positive case location attributes to determine the location risk factor.

4. The computer-implemented method of claim 1, wherein determining the individual risk factor further comprises:
assigning, by one or more processors, an individual attribute risk level score to each of the individual attributes;
generating, by one or more processors, a weighted attribute risk level score by applying an attribute weight to each corresponding individual attribute; and
aggregating, by one or more processors, the weighted attribute risk level score for each of the individual attributes to determine the individual risk factor.

5. The computer-implemented method of claim 2, further comprising:
applying, by one or more processors, a time weight to the local individual risk level, wherein the time weight is directly proportional to a time duration the individual is present at the location.

6. The computer-implemented method of claim 2, further comprising:
determining, by one or more processors, a first unified risk factor for the individual at the location based on the individual risk factor and the location risk factor.

7. The computer-implemented method of claim 6, further comprising:
detecting, by one or more processors, a change in one or more of the positive case location attributes and the individual attributes that exceeds a predetermined threshold;
responsive to detecting the change, determining, by one or more processors, a second unified risk factor for the individual at the location based on the individual risk factor and the location risk factor; and
responsive to the second unified risk factor satisfying the condition, generating, by one or more processors, the alert for one or more of the location and the individual.

8. A computer program product, comprising:
one or more computer readable storage media and program instructions stored on the one or more computer readable storage media, the program instructions comprising:
program instructions to receive individual profile data comprising individual attributes corresponding to an individual, the individual attributes comprising demographic data, medical data, and individual location data;
program instructions to determine an individual risk factor based at least on the individual profile data; and
responsive to the individual risk factor satisfying a first condition, program instructions to generate a first alert indicating the individual risk factor for the individual.

9. The computer program product of claim 8, further comprising:
program instructions to receive location data corresponding to a positive case at a location, the location data comprising positive case location attributes comprising a local individual risk level, a location function, location size, location surface contact frequency, and location air access;
program instructions to determine a location risk factor for the location based at least on the location data; and
responsive to the location risk factor satisfying a second condition, program instructions to generate a second alert indicating the location risk factor for the location.

10. The computer program product of claim 9, wherein the program instructions to determine the location risk factor further comprises:
program instructions to assign a location risk level score to each of the positive case location attributes;
program instructions to generate a weighted location risk level score by applying a location factor weight to each corresponding positive case location attribute; and
program instructions to aggregate the weighted location risk level score for each of the positive case location attributes to determine the location risk factor.

11. The computer program product of claim 8, wherein the program instructions to determine the individual risk factor further comprises:
program instructions to assign an individual attribute risk level score to each of the individual attributes;
program instructions to generate a weighted attribute risk level score by applying an attribute weight to each corresponding individual attribute; and
program instructions to aggregate the weighted attribute risk level score for each of the individual attributes to determine the individual risk factor.

12. The computer program product of claim 9, further comprising:
program instructions to apply a time weight to the local individual risk level, wherein the time weight is directly proportional to a time duration the individual is present at the location.

13. The computer program product of claim 9, further comprising:
program instructions to determine a first unified risk factor for the individual at the location based on the individual risk factor and the location risk factor.

14. The computer program product of claim 13, further comprising:
program instructions to detect a change in one or more of the positive case location attributes and the individual attributes that exceeds a predetermined threshold;
responsive to the program instructions to detect the change, program instructions to determine a second unified risk factor for one or more of the individual risk factor and the location risk factor; and
responsive to the second unified risk factor satisfying the condition, program instructions to generate the alert for one or more of the location and the individual.

15. A computer system, comprising:
one or more computer processors;
one or more computer readable storage media;
program instructions stored on the one or more computer readable storage media for execution by at least one of the one or more processors, the program instructions comprising:
program instructions to receive individual profile data comprising individual attributes corresponding to an individual, the individual attributes comprising demographic data, medical data, and individual location data;
program instructions to determine an individual risk factor based at least on the individual profile data; and
responsive to the individual risk factor satisfying a first condition, program instructions to generate a first alert indicating the individual risk factor for the individual.

16. The computer system of claim 15, further comprising:
program instructions to receive location data corresponding to a positive case at a location, the location data comprising positive case location attributes comprising a local individual risk level, a location function, location size, location surface contact frequency, and location air access;
program instructions to determine a location risk factor for the location based at least on the location data; and
responsive to the location risk factor satisfying a second condition, program instructions to generate a second alert indicating the location risk factor for the location.

17. The computer system of claim 16, wherein the program instructions to determine the location risk factor further comprises:
program instructions to assign a location risk level score to each of the positive case location attributes;
program instructions to generate a weighted location risk level score by applying a location factor weight to each corresponding positive case location attribute; and
program instructions to aggregate the weighted location risk level score for each of the positive case location attributes to determine the location risk factor.

18. The computer system of claim 15, wherein the program instructions to determine the individual risk factor further comprises:
program instructions to assign an individual attribute risk level score to each of the individual attributes;
program instructions to generate a weighted attribute risk level score by applying an attribute weight to each corresponding individual attribute; and
program instructions to aggregate the weighted attribute risk level score for each of the individual attributes to determine the individual risk factor.

19. The computer system of claim 16, further comprising:
program instructions to determine a first unified risk factor for the individual at the location based on the individual risk factor and the location risk factor; and
program instructions to apply a time weight to the local individual risk level, wherein the time weight is directly proportional to a time duration the individual is present at the location.

20. The computer system of claim 16, further comprising:
program instructions to detect a change in one or more of the positive case location attributes and the individual attributes that exceeds a predetermined threshold;
responsive to the program instructions to detect the change, program instructions to determine a second unified risk factor for one or more of the individual risk factor and the location risk factor; and
responsive to the second unified risk factor satisfying the condition, program instructions to generate the alert for one or more of the location and the individual.
